(19) United States
(12) Patent Application Publication
(10) Pub. No.: US 2026/0102062 A1
(43) Pub. Date: Apr. 16, 2026

(54) INTELLIGENT OPTOMETRY AND GLASSES FITTING ASSISTANCE SYSTEM AND METHOD FOR ORTHOKERATOLOGY LENSES BASED ON CORNEAL TOPOGRAPHY

(71) Applicant: BRIGHTEN OPTIX CORP., Taipei City (TW)

(72) Inventors: Wen-Kai LI, Taipei City (TW); I-Tsung WU, Taipei City (TW); Wen-Pin LIN, Taipei City (TW)

(21) Appl. No.: 18/912,880

(22) Filed: Oct. 11, 2024

(51) Int. Cl. A61B 3/00; A61B 3/107; G02C 7/02

(52) U.S. Cl. CPC A61B 3/0025; A61B 3/107; G02C 7/027

(57) ABSTRACT

An intelligent optometry and glasses fitting assistance system and method for orthokeratology lenses based on corneal topography, includes a detection module to obtain patient's optometry parameter and corneal characteristic parameter, a corneal topography preliminary module to draw a corneal preliminary topography map based on the optometry parameter and the corneal characteristic parameter, a corneal topography optimization module to obtain the corneal optimized topography map based on the corneal preliminary topography map, a model construction module to build a corneal reshaping lens evaluation auxiliary model based on the predetermined corneal reshaping lens parameter combined with the optometry parameter, corneal characteristic parameter and corneal optimized topography map, and a model evaluation module to evaluate the corneal reshaping lens evaluation auxiliary model based on a predetermined evaluation index. The evaluation calculation method calculates the contact area between the lens and the cornea and outputs the best corneal reshaping lens parameter.

### BACKGROUND OF THE INVENTION

### 1. Field of the Invention

The invention provides an intelligent optometry and glasses fitting assistance system and method for orthokeratology lenses based on corneal topography, in particular, it provides a corneal reshaping lens evaluation auxiliary model to calculate a lens alignment zone contact area AZ of the corneal reshaping lens parameter, and an implementable range of the lens alignment zone contact area AZ is between (DIA-C1)/2 and (DIA-OZ-C2)/2. The above formula is based on the ophthalmologists or optometrists'pre-setting of the patient's basic optometry parameters in the optometry and glasses fitting assistance system, which can eliminate the human uncertainty factor of the ophthalmologists or optometrists directly evaluating and trying glasses on patients multiple times. Through the approximation algorithm, the ophthalmologists or optometrists can quickly obtain the appropriate corneal reshaping lens parameters according to the set constraints and target function. Compared with the traditional manual optometry and glasses fitting method, more accurate corneal reshaping lens parameters can be obtained at one time, and prefer vision control results are expected.

### 2. Description of the Related Art

Orthokeratology lenses are mainly used for children, adolescents or young adults before the age of 18 whose vision has not yet been established. The orthokeratology lenses are similar in structure to hard contact lenses and are worn at night while sleeping. The method of controlling vision is to first fit the peripheral positioning area of the orthokeratology lens to the eyeball, and then use the flatter area in the center of the orthokeratology lens to compress the center of the cornea. The reversal arc located at the outer edge of the center of the orthokeratology lens is used to accommodate the corneal epidermis that migrates to the sides, thereby realigning the corneal epidermis and making the center flatter and the periphery steeper, thereby achieving the purpose of controlling vision.

It is known that orthokeratology lenses need to be evaluated and tried on by an ophthalmologist or optometrist for multiple times before the optimal orthokeratology lens parameters can be determined and the orthokeratology lenses can be tailor-made for the patient. However, the above model is highly dependent on the skills and experience of the ophthalmologist or optometrist himself. If the ophthalmologist or optometrist has poor skills and experience, the patient will not be able to obtain suitable orthokeratology lenses, and the results of controlling vision will not be apparent. The problems caused by learning and skills need to be improved and solved by those engaged in this industry. As for the problems arising from the prior art, it is up to those working in this industry to improve and solve them.

### SUMMARY OF THE INVENTION

Therefore, in view of the above-mentioned problems and deficiencies, the inventor collected relevant information, and after many evaluations and considerations, he designed the invention of this intelligent optometry and glasses fitting assistance system and method for orthokeratology lenses based on corneal topography

The main object of the present invention is to provide an intelligent optometry and glasses fitting assistance system and method for orthokeratology lenses based on corneal topography. The intelligent optometry and glasses fitting assistance system comprises an optometry and glasses fitting assistance system, and a detection module used to obtain an optometry parameter and a corneal characteristic parameter of the patient. The optometry and glasses fitting assistance system comprises a corneal topography preliminary module, a corneal topography optimization module, a model construction module, and a model evaluation module. The corneal topography preliminary module is provided for drawing a corneal preliminary topography map according to the optometry parameter and the corneal characteristic parameter. The corneal topography optimization module is provided for performing reconstruction, optimization adjustment and resampling according to the corneal preliminary topography map to obtain a corneal optimized topography map. The model construction module is provided for obtaining a corneal reshaping lens evaluation auxiliary model according to a predetermined corneal reshaping lens parameter combined with the optometry parameter, the corneal characteristic parameter and the corneal optimized topography map. The corneal reshaping lens evaluation auxiliary model calculates a lens alignment zone contact area AZ of the corneal reshaping lens parameter. The implementable range of the lens alignment zone contact area AZ is between (DIA-C1)/2 and (DIA-OZ-C2)/2, wherein DIA is the lens diameter; OZ is the lens optical zone contact area, which is provided by the corneal reshaping lens parameter; C1 is the first calculation parameter; and C2 is the second calculation parameter. The model evaluation module is provided for performing evaluation calculation on the corneal reshaping lens evaluation auxiliary model according to a predetermined evaluation index, wherein the evaluation calculation method is to calculate the contact area between the lens and the cornea, and output an evaluation result.

The lens alignment zone contact area AZ of the corneal reshaping lens parameter is calculated by using the above-mentioned corneal reshaping lens evaluation auxiliary model, and the implementable range of the lens alignment zone contact area AZ is between (DIA-C1)/2 and (DIA-OZ-C2)/2. The above formula is based on the patient's basic optometry parameter pre-set by ophthalmologists or optometrists in the optometry and glasses fitting assistance system, which can eliminate the human uncertainty factor of the ophthalmologists or optometrists directly evaluating and trying glasses on patients multiple times. Through the approximation algorithm, the ophthalmologists or optometrists can quickly obtain the appropriate corneal reshaping lens parameter according to the set constraints and target function. Compared with the traditional manual optometry and glasses fitting method, more accurate corneal reshaping lens parameters can be obtained at one time, and prefer visual control results are expected.

Another object of the present invention is that the optimization adjustment of the corneal topography optimization module is to establish an optimization function, and the formula for obtaining the value of the optimization function is: ∫w1×OZ−w2×AZ; wherein w1 is the first weighting factor; w2 is the second weighting factor; OZ is the lens optical zone contact area (Optical Zone); AZ is the lens alignment zone contact area (Alignment Zone), the value of the optimization function being able to be linear programming (LP), mixed integer linear programming (MILP), quadratic programming (QP), second-order pyramid programming (SOCP), nonlinear programming (NLP), constrained linear least squares, nonlinear least squares and nonlinear equations.

Still another object of the present invention is that the parameter range of C1 is between 0.5 mm and 1.5 mm, and the initial value is 0.8 mm.

Still another object of the present invention is that the parameter range of C2 is between 1 mm and 2 mm, and the initial value is 1.2 mm.

Still another object of the present invention is that the prefer operation of the corneal reshaping lens evaluation auxiliary model is to obtain the satisfied constraints and the minimized or maximized target parameters through the approximation algorithm, and the method for obtaining the minimized target parameters of the point is to make the first-order derivative be zero (f′(x)=0), and if the second-order derivative of the point is positive (f′(x) [symbol] 0), then the point is the local minimum, and the method for obtaining the maximized target parameter is to make the first-order derivative of the point be zero (f′(x)=0), and if the second-order derivative of the point is negative (f″(x) [symbol] 0), then the point is the local maximum.

### BRIEF DESCRIPTION OF THE DRAWINGS

FIG. 1 is a functional block diagram of the intelligent optometry and glasses fitting assistance system of the present invention.

FIG. 2 is a proportional diagram of the initial corneal topography of the present invention.

FIG. 3 is a proportional diagram of the corneal optimized topography of the present invention.

FIG. 4 is a dimensional diagram of the interaction model of the lens and cornea of the present invention.

FIG. 5 is a dimensional diagram of the fluorescent tear fluid model of the present invention.

FIG. 6 is a flowchart of the steps of the intelligent optometry and glasses fitting assistance method of the present invention.

### DETAILED DESCRIPTION OF THE INVENTION

In order to achieve the above objects and effects, the technical means and structures adopted by the present invention are described in detail below with respect to the preferred embodiment of the present invention, so as to facilitate a complete understanding.

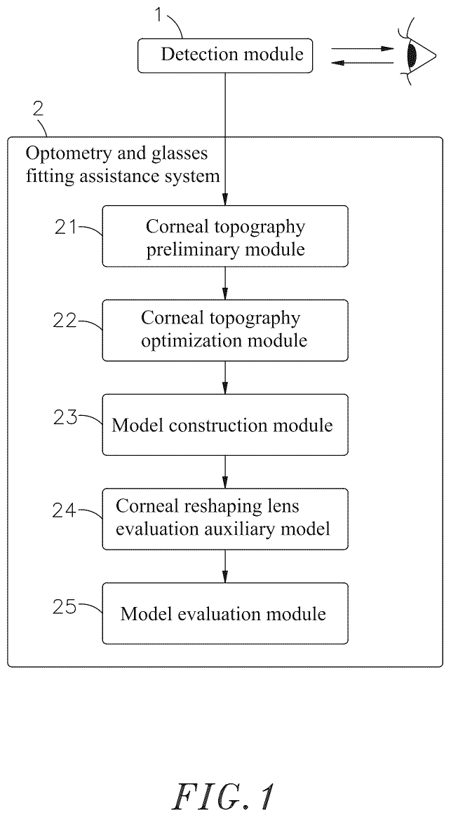

Please refer to FIGS. 1 to 5, which are a functional block diagram of the intelligent optometry and glasses fitting assistance system of the present invention, a proportional diagram of the initial corneal topography of the present invention, a proportional diagram of the corneal optimized topography of the present invention, a dimensional diagram of the interaction model of the lens and cornea of the present invention and a dimensional diagram of the fluorescent tear fluid model of the present invention. As can be clearly seen from the figures, the intelligent optometry and glasses fitting assistance system of the present invention mainly comprises: a detection module 1 and an optometry and glasses fitting assistance system 2. The optometry and glasses fitting assistance system 2 refers to a personal computer (PC), a notebook computer (Notebook), a tablet computer (Table PC) or a smartphone. The optometry and glasses fitting assistance system 2 has a storage device (not shown in the figure, such as a hard disk drive (HDD), a solid state drive (SDD) or a non-volatile flash memory, and its main components and features are described in detail as follows:

The detection module 1 is used to obtain an optometry parameter and a corneal characteristic parameter 4 of the patient.

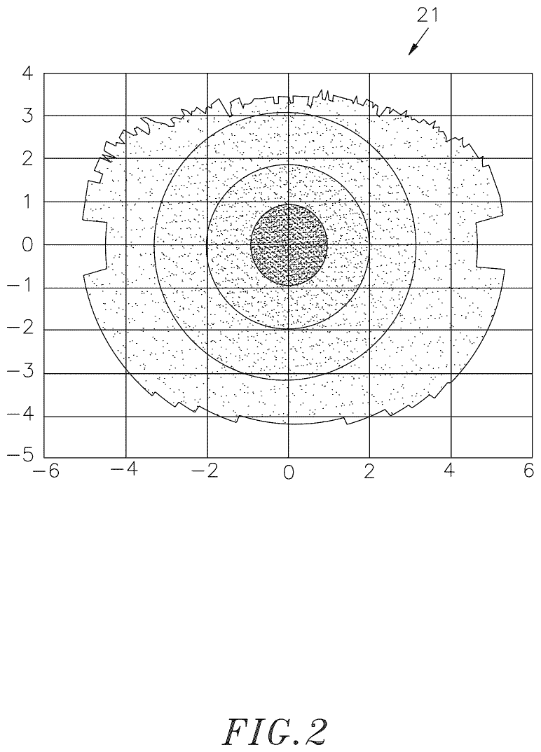

A corneal topography preliminary module 21 is provided in the optometry and glasses fitting assistance system 2, and draws a corneal preliminary topography map (as shown in FIG. 2) according to the optometry parameter and the corneal characteristic parameter 4.

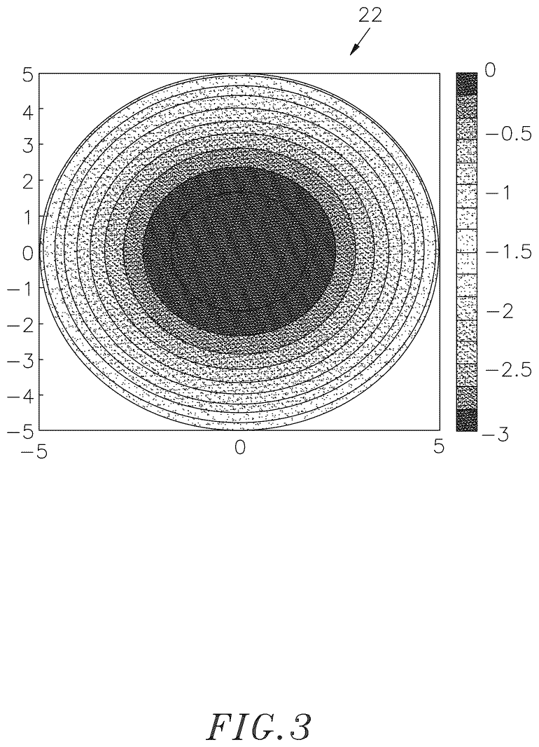

A corneal topography optimization module 22 is provided in the optometry and glasses fitting assistance system 2, and performs reconstruction, optimization adjustment and resampling according to the corneal preliminary topography map to obtain a corneal optimized topography map (as shown in FIG. 3).

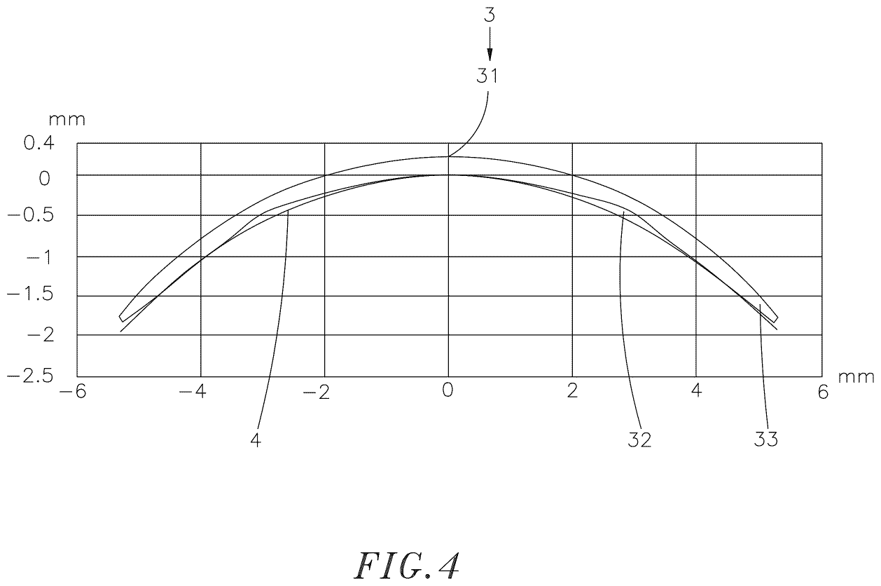

A model construction module 23 is provided in the optometry and glasses fitting assistance system 2, and obtains a corneal reshaping lens evaluation auxiliary model 24 according to a predetermined corneal reshaping lens parameter (as shown in FIG. 4) combined with the optometry parameter, the corneal characteristic parameter 4 (as shown in FIG. 4) and the corneal optimized topography map.

The corneal reshaping lens evaluation auxiliary model 24 calculates a lens alignment zone contact area AZ of the corneal reshaping lens parameter, and an implementable range of the lens alignment zone contact area AZ is between (DIA-C1)/2 and (DIA-OZ-C2)/2, where DIA is the lens diameter; OZ is the lens optical zone contact area, which is provided by the corneal reshaping lens parameter; C1 is the first calculation parameter; and C2 is the second calculation parameter.

A model evaluation module 25 is provided in the optometry and glasses fitting assistance system 2, and performs an evaluation calculation on the corneal reshaping lens evaluation auxiliary model 24 according to a predetermined evaluation index, wherein the evaluation calculation method is to calculate the contact area between the lens and the cornea, and output an evaluation result.

The optimization adjustment of the corneal topography optimization module 22 is to establish an optimization function, and the formula for obtaining the value of the optimization function is: ∫w1×OZ−w2×AZ; wherein w1 is the first weighting factor; w2 is the second weighting factor; OZ is the lens optical zone contact area (Optical Zone); AZ is the lens alignment zone contact area (Alignment Zone). W1 and W2 are weighted according to the material of the orthokeratology lens, ambient temperature and ambient humidity. The value of the optimization function can be linear programming (LP), mixed integer linear programming (MILP), quadratic programming (QP), second-order pyramid programming (SOCP), nonlinear programming (NLP), constrained linear least squares, nonlinear least squares and nonlinear equations.

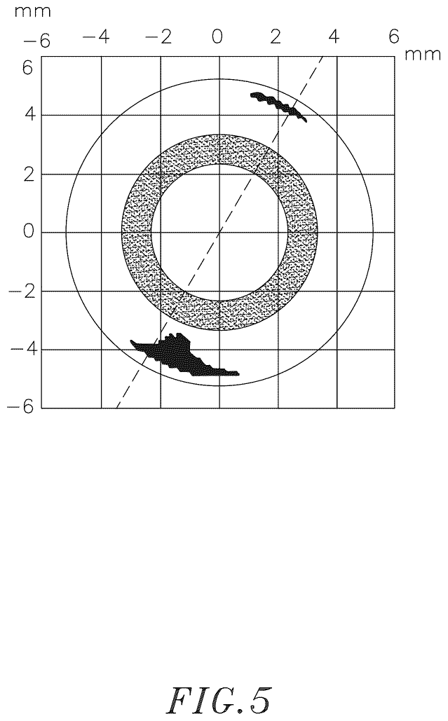

As shown in FIGS. 4 and 5, the corneal reshaping lens evaluation auxiliary model 24 comprises a lens and corneal interaction model and a fluorescent tear fluid model. The corneal reshaping lens parameter 3 is obtained by selecting the closest parameter between the optometry parameter and the corneal characteristic parameter 4. The corneal reshaping lens parameter 3 further comprises a lens optical zone 31, a lens reversal arc 32 and a lens alignment zone 33. The method of controlling vision is to first make the lens alignment zone 33 fit the corneal characteristic parameter 4, and then compress the center of the corneal characteristic parameter 4 through the lens optical zone 31. The lens reversal arc 32 located at the outer edge of the lens optical zone 31 is used to collect the corneal epidermis and tears moving to the sides, so that the purpose of controlling vision is achieved by realigning the corneal epidermis and making the center flatter and the periphery steeper.

The parameter range of C1 is between 0.5 mm and 1.5 mm, and the initial value is 0.8 mm; and the parameter range of C2 is between 1 mm and 2 mm, and the initial value is 1.2 mm. C1 and C2 are adjusted according to the sizes of the lens optical zone 31, the lens reversal arc 32 and the lens alignment zone 33 of the corneal reshaping lens parameter 3.

The prefer operation of the above-mentioned corneal reshaping lens evaluation auxiliary model 24 is to obtain the satisfied constraints and the minimized or maximized target parameters through the approximation algorithm, and the method for obtaining the minimized target parameter is to make the first-order derivative of the point be zero (f′(x)=0). If the second-order derivative of the point is positive (f″(x) [symbol] 0), then the point is the local minimum. The method for obtaining the maximized target parameter is to make the first-order derivative of the point be zero (f′(x)=0). If the second-order derivative of the point is negative (f″(x) [symbol] 0), then the point is the local maximum.

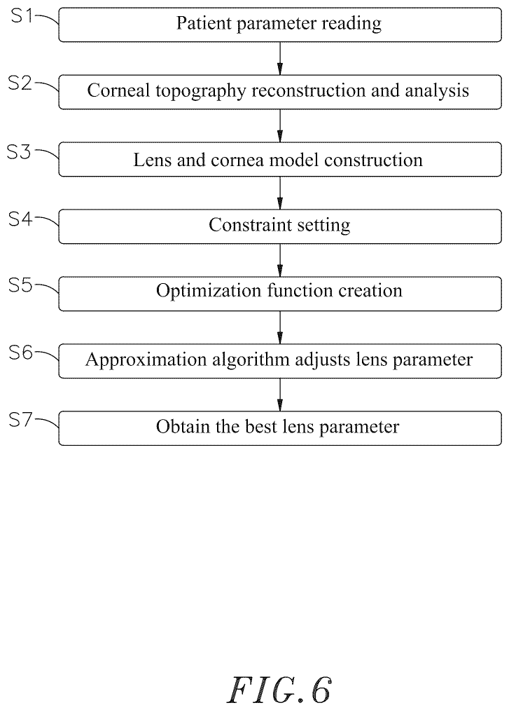

Please refer to FIG. 6, which is a flowchart of the steps of the intelligent optometry and glasses fitting assistance method of the present invention, comprising:
Step S1: Patient parameter reading, which is to provide a detection module to obtain an optometry parameter and a corneal characteristic parameter of the patient through detection.
Step S2: Corneal topography reconstruction and analysis, which is to provide a corneal topography preliminary module to draw a corneal preliminary topography map according to the optometry parameter and the corneal characteristic parameter, and to provide a corneal topography optimization module to perform reconstruction, optimization adjustment and resampling based on the corneal preliminary topography map to obtain a corneal optimized topography map.
Step S3: Lens and cornea model construction, which is to provide a model construction module to obtain a corneal reshaping lens evaluation auxiliary model according to a predetermined corneal reshaping lens parameter combined with the optometry parameter, the corneal characteristic parameter and the corneal optimized topography map, wherein the corneal reshaping lens parameter is obtained by selecting the parameter that is closest to the optometry parameter and the corneal characteristic parameter; and the corneal reshaping lens evaluation auxiliary model comprises a lens and corneal interaction model and a fluorescent tear fluid model.
Step S4: Constraint setting, which is to set the number of iterations of the corneal reshaping lens evaluation auxiliary model, and calculate a lens alignment zone contact area AZ of the corneal reshaping lens parameter, and the implementable range of the lens alignment zone contact area AZ is between (DIA-C1)/2 and (DIA-OZ-C2)/2, where DIA is the lens diameter, OZ is the lens optical zone contact area, which is provided by the corneal reshaping lens parameter, C1 is the first calculation parameter, and C2 is the second calculation parameter; the parameter range of C1 is between 0.5 mm and 1.5 mm, and the initial value is 0.8 mm; and the parameter range of C2 is between 1 mm and 2 mm, and the initial value is 1.2 mm.
Step S5: Optimization function creation, which is to provide a model evaluation module to perform an evaluation calculation on the corneal reshaping lens evaluation auxiliary model according to a predetermined evaluation index, and the evaluation calculation method calculates the contact area between the lens and the cornea, and outputs an evaluation result, wherein the optimization adjustment of the corneal topography optimization module is to establish an optimization function, and the formula for obtaining the value of the optimization function is: ∫w1×OZ−w2×AZ; wherein w1 is the first weighting factor; w2 is the second weighting factor; OZ is the lens optical zone contact area (Optical Zone); AZ is the lens alignment zone contact area (Alignment Zone).

Step S6: Approximation algorithm adjusts lens parameter, where the approximation algorithm obtains the satisfied constraints and the minimized or maximized target parameters; the method for obtaining the minimized target parameters is to make the first-order derivative of the point be zero (f′(x)=0), and if the second-order derivative of the point is positive (f″(x) [symbol] 0), then the point is the local minimum; the method for obtaining the maximized target parameter is to make the first-order derivative of the point be zero (f′(x)=0), and if the second-order derivative of the point is negative (f″(x) [symbol] 0), then the point is the local maximum.

Step S7: Obtain the best lens parameter.

The main features of the present invention are: providing a corneal reshaping lens evaluation auxiliary model 24 to calculate the lens alignment zone contact area AZ of the corneal reshaping lens parameter 3. The implementable range of the lens alignment zone contact area AZ is between (DIA-C1)/2 and (DIA-OZ-C2)/2. The above formula is based on the patient's basic optometry parameters pre-set by ophthalmologists or optometrists in the optometry and glasses fitting assistance system 2, which can eliminate the human uncertainty factor of the ophthalmologists or optometrists directly evaluating and trying glasses on patients multiple times. Through the approximation algorithm, the ophthalmologists or optometrists can quickly obtain the appropriate corneal reshaping lens parameter 3 according to the set constraints and target function. Compared with the traditional manual optometry and glasses fitting method, more accurate corneal reshaping lens parameters 3 can be obtained at one time, and prefer visual control results are expected.

The above description is only a preferred embodiment of the present invention and does not limit the patent scope of the present invention. Therefore, all simple modifications and equivalent structural changes made by using the description and drawings of the present invention shall be included in the patent scope of the present invention and shall be clearly stated.

In summary, the above-mentioned intelligent optometry and glasses fitting assistance system and method for orthokeratology lenses based on corneal topography of the present invention can indeed achieve its effect and purpose when used. Therefore, this invention is truly an invention with excellent practicality and meets the application requirements for an invention patent, so the application must be filed in accordance with the law. We hope that the review committee will approve this case as soon as possible to protect the inventor's hard work. If the review committee has any doubts, please feel free to send us a letter for instructions. The inventor will do our best to cooperate. We sincerely appreciate it.

What is claimed is:

1. An intelligent optometry and glasses fitting assistance system for orthokeratology lenses based on corneal topography, comprising an optometry and glasses fitting assistance system and a detection module used to obtain an optometry parameter and a corneal characteristic parameter of the patient, wherein, said optometry and glasses fitting assistance system comprises:
a corneal topography preliminary module provided for drawing a corneal preliminary topography map according to said optometry parameter and said corneal characteristic parameter;
a corneal topography optimization module provided for performing reconstruction, optimization adjustment and resampling according to said corneal preliminary topography map to obtain a corneal optimized topography map;
a model construction module provided for obtaining a corneal reshaping lens evaluation auxiliary model according to a predetermined corneal reshaping lens parameter combined with said optometry parameter, said corneal characteristic parameter and said corneal optimized topography map, said corneal reshaping lens evaluation auxiliary model calculating a lens alignment zone contact area AZ of said corneal reshaping lens parameter, the implementable range of said lens alignment zone contact area AZ being between (DIA-C1)/2 and (DIA-OZ-C2)/2; wherein DIA is the lens diameter; OZ is the lens optical zone contact area, which is provided by said corneal reshaping lens parameter; C1 is the first calculation parameter; and C2 is the second calculation parameter; and
a model evaluation module provided for performing evaluation calculation on said corneal reshaping lens evaluation auxiliary model according to a predetermined evaluation index, wherein the evaluation calculation method is to calculate the contact area between the lens and the cornea, and output an evaluation result.

2. The intelligent optometry and glasses fitting assistance system for orthokeratology lenses based on corneal topography as claimed in claim 1, wherein the optimization adjustment of said corneal topography optimization module is to establish an optimization function, and the formula for obtaining the value of the optimization function is: ∫w1×OZ−w2×AZ; wherein w1 is the first weighting factor; w2 is the second weighting factor; OZ is the lens optical zone contact area (Optical Zone); AZ is the lens alignment zone contact area (Alignment Zone), the value of the optimization function being able to be linear programming (LP), mixed integer linear programming (MILP), quadratic programming (QP), second-order pyramid programming (SOCP), nonlinear programming (NLP), constrained linear least squares, nonlinear least squares and nonlinear equations.

3. The intelligent optometry and glasses fitting assistance system for orthokeratology lenses based on corneal topography as claimed in claim 1, wherein said corneal reshaping lens parameter is obtained by selecting the parameter that is closest to said optometry parameter and said corneal characteristic parameter.

4. The intelligent optometry and glasses fitting assistance system for orthokeratology lenses based on corneal topography as claimed in claim 1, wherein said corneal reshaping lens evaluation auxiliary model comprises a lens and corneal interaction model and a fluorescent tear fluid model.

5. The intelligent optometry and glasses fitting assistance system for orthokeratology lenses based on corneal topography as claimed in claim 1, wherein the parameter range of C1 is between 0.5 mm and 1.5 mm, and the initial value is 0.8 mm.

6. The intelligent optometry and glasses fitting assistance system for orthokeratology lenses based on corneal topography as claimed in claim 1, wherein the parameter range of C2 is between 1 mm and 2 mm, and the initial value is 1.2 mm.

7. The intelligent optometry and glasses fitting assistance system for orthokeratology lenses based on corneal topography as claimed in claim 1, wherein the prefer operation of said corneal reshaping lens evaluation auxiliary model is to obtain the satisfied constraints and the minimized or maximized target parameters through the approximation algorithm, and the method for obtaining the minimized target parameter is to make the first-order derivative of the point be zero (f′(x)=0), and if the second-order derivative of the point is positive (f″(x) [symbol] 0), then the point is the local minimum, and the method for obtaining the maximized target parameter is to make the first-order derivative of the point be zero (f′(x)=0), and if the second-order derivative of the point is negative (f″(x) [symbol] 0), then the point is the local maximum.

8. An intelligent optometry and glasses fitting assistance method for orthokeratology lenses based on corneal topography, comprising the steps of:
Step S1: Patient parameter reading, which is to provide a detection module to obtain an optometry parameter and a corneal characteristic parameter of the patient through detection;
Step S2: Corneal topography reconstruction and analysis, which is to provide a corneal topography preliminary module to draw a corneal preliminary topography map according to said optometry parameter and said corneal characteristic parameter, and to provide a corneal topography optimization module to perform reconstruction, optimization adjustment and resampling based on said corneal preliminary topography map to obtain a corneal optimized topography map;
Step S3: Lens and cornea model construction, which is to provide a model construction module to obtain a corneal reshaping lens evaluation auxiliary model according to a predetermined corneal reshaping lens parameter combined with said optometry parameter, said corneal characteristic parameter and said corneal optimized topography map;
Step S4: Constraint setting, which is to set the number of iterations of said corneal reshaping lens evaluation auxiliary model, and calculate a lens alignment zone contact area AZ of said corneal reshaping lens parameter, and the implementable range of said lens alignment zone contact area AZ is between (DIA-C1)/2 and (DIA-OZ-C2)/2, wherein DIA is the lens diameter, OZ is the lens optical zone contact area, which is provided by said corneal reshaping lens parameter, C1 is the first calculation parameter, and C2 is the second calculation parameter;
Step S5: Optimization function creation, which is to provide a model evaluation module to perform an evaluation calculation on said corneal reshaping lens evaluation auxiliary model according to a predetermined evaluation index, and the evaluation calculation method calculates the contact area between the lens and the cornea, and outputs an evaluation result;
Step S6: Approximation algorithm adjusting lens parameter; and
Step S7: Obtaining the best lens parameter.

9. The intelligent optometry and glasses fitting assistance method for orthokeratology lenses based on corneal topography as claimed in claim 8, wherein in Step S5, the formula for obtaining the value of the optimization function is: ∫w1×OZ−w2×AZ; wherein w1 is the first weighting factor; w2 is the second weighting factor; OZ is the lens optical zone contact area (Optical Zone); AZ is the lens alignment zone contact area (Alignment Zone), said optimization function being able to be linear programming (LP), mixed integer linear programming (MILP), quadratic programming (QP), second-order pyramid programming (SOCP), nonlinear programming (NLP), constrained linear least squares, nonlinear least squares and nonlinear equations.

10. The intelligent optometry and glasses fitting assistance method for orthokeratology lenses based on corneal topography as claimed in claim 8, wherein in Step S3, said corneal reshaping lens parameter is obtained by selecting the parameter that is closest to said optometry parameter and said corneal characteristic parameter.

11. The intelligent optometry and glasses fitting assistance method for orthokeratology lenses based on corneal topography as claimed in claim 8, wherein in Step S3, said corneal reshaping lens evaluation auxiliary model comprises a lens and corneal interaction model and a fluorescent tear fluid model.

12. The intelligent optometry and glasses fitting assistance method for orthokeratology lenses based on corneal topography as claimed in claim 8, wherein in Step S4, the parameter range of C1 is between 0.5 mm and 1.5 mm, and the initial value is 0.8 mm.

13. The intelligent optometry and glasses fitting assistance method for orthokeratology lenses based on corneal topography as claimed in claim 8, wherein in Step S4, the parameter range of C2 is between 1 mm and 2 mm, and the initial value is 1.2 mm.

14. The intelligent optometry and glasses fitting assistance method for orthokeratology lenses based on corneal topography as claimed in claim 8, wherein in Step S6, the approximation algorithm obtains the satisfied constraints and the minimized or maximized target parameters, and the method for obtaining the minimized target parameter is to make the first-order derivative of the point be zero (f′(x)=0), and if the second-order derivative of the point is positive (f″(x) [symbol] 0), then the point is the local minimum, and the method for obtaining the maximized target parameter is to make the first-order derivative of the point be zero (f′(x)=0), and if the second-order derivative of the point is negative (f″(x) [symbol] 0), then the point is the local maximum.
